(19) United States
(12) Patent Application Publication
(10) Pub. No.: US 2024/0238004 A1
(43) Pub. Date: Jul. 18, 2024

(54) CIRCUMFERENTIAL NERVE CUTTER

(71) Applicant: CHECKPOINT SURGICAL, INC., Independence, OH (US)

(72) Inventors: Derek LEWIS, Cleveland, OH (US); Trevor KUCHERAK, Cleveland, OH (US); Benjamin COTTRILL, Cleveland, OH (US); Eric WALKER, Cleveland, OH (US); Brandon S. SMETANA, Cleveland, OH (US)

(21) Appl. No.: 18/576,614

(22) PCT Filed: Jul. 19, 2022
(86) PCT No.: PCT/US2022/037580

§ 371 Date: Jan. 4, 2024

Related U.S. Application Data
(60) Provisional application No. 63/223,338, filed on Jul. 19, 2021.

(51) Int. Cl. A61B 17/3211; A61B 17/32

(52) U.S. Cl. CPC A61B 17/3211; A61B 2017/320052

(57) ABSTRACT

A circumferential nerve cutter is described herein. The nerve cutter holds a nerve in tension while circumferentially constraining the side of the nerve being retained in a manner that prevents flattening or crushing. A head of the nerve cutter separates the preserved side from the portion of the nerve being cut that is being removed. The head includes a cutting slot configured to accommodate an off-the-shelf scalpel blade, while ensuring that the blade remains approximately square preventing obliquity of resultant cut. The side of the head that interfaces with the preserved side of the nerve is configured to prevent and/or inhibit the nerve from moving radially to its axis while being cut.

### CROSS REFERENCE TO RELATED APPLICATIONS

This application claims priority to U.S. Patent Application Ser. No. 63/223,338 filed on Jul. 19, 2021 and entitled “Circumferential Nerve Cutter,” which is incorporated herein by reference.

### TECHNICAL FIELD

The present disclosure generally relates to non-powered surgical tools and, more specifically, a circumferential nerve cutter.

### BACKGROUND

A transection or partial resection of a nerve, sometimes referred to as a neurectomy, may be performed for pain management, during the surgical repair of a damaged nerve or for various other medical reasons. Traditionally, such resections are performed using surgical scissors or a scalpel, with or without the use of additional tools such as one acting as a solid background (e.g. a tongue depressor) or tendon holding forceps to assist with the resection. However, use of scissors or a scalpel with solid background may crush all or portions of the nerve end and/or cause uneven cuts. This may result in poorly aligned nerve structures at a coaptation site and/or difficulty in aligning the nerve ends to perform the coaptation. Tendon holding or vasectomy forceps facilitate cutting perpendicular to the axis of the nerve. However, the forceps may slide axially along the nerve when they are sized not to crush the nerve. Thus, when sized in this manner, cutting the nerve under tension requires an additional instrument to provide tension axially to the nerve. This may require multiple users, one user tensioning the nerve axially using tweezer or forceps with another user holding the tendon forceps to resect the nerve using a scalpel. Sometimes these forceps use a proprietary blade that presents supply stocking constraints and maintenance of a proprietary blade holder. Slotted instruments require diligence in cleaning so as not to bend the instrument in a way that narrows or widens a blade guide slot. Some nerve cutting forceps include a relatively long jaw with multiple apertures to provide a one size fits most tool. However, this requires a longer in-wound instrument head.

There is, therefore, a need for an improved nerve cutting tool that holds the nerve in tension while circumferentially constraining the preserved side in a manner that prevents flattening or crushing while facilitating performance of the nerve resection with two hands.

### SUMMARY

A circumferential nerve cutter is described herein that is configured to hold a nerve in a tensioned position while circumferentially constraining the side of the nerve being retained (sometimes referred to as the “preserved side of the nerve”) in a manner that prevents flattening or crushing of the nerve. The circumferential nerve cutter facilitates performing the nerve resection where the nerve is tensioned, circumferentially restrained, and cut. In some examples, the circumferential nerve cutter may be operated by one clinician, while another clinician operates the cutting instrument. A head of the circumferential nerve cutter separates the retained or preserved side from the portion of the nerve being cut that is being removed (sometimes referred to as the “sacrificial side of the nerve”). The head includes a cutting slot configured to accommodate a cutting blade, often a typical surgical scalpel, while ensuring that the blade remains approximately square preventing obliquity of resultant cut. The side of the head that interfaces with the preserved side of the nerve (e.g., the “preserved side”) is configured to prevent and/or inhibit the nerve from moving radially to its axis while being cut without pinching or crushing. The other side of the head engages and holds the portion of the nerve being cut that is being removed (sometimes referred to as the “sacrificial side of the nerve”). In some examples, the circumferential nerve cutter includes a retaining mechanism such as a ratchet and pawl mechanism holding the cutter head to a fixed opening aperture to facilitate an interval of other activities between cutter placement and cutting activity. In some examples, the circumferential nerve cutter includes an integrated sizing guide for selection of the appropriate circumferential nerve cutter from a range of differing size options. The sizing guide is configured to minimize the required thickness such that the sizing operation requires minimal lifting of the nerve within the surgical wound. In some examples, a smaller sizing guide cutout is located at the tip of the circumferential nerve cutter, the larger sizing guide cutout is located inside proximate the smaller sizing guide cutout, stemming off of the curved edge. This curved end of the circumferential nerve cutter facilitates a “hooking under” the nerve.

A nerve cutter may comprise a head having an upper portion and a separate lower portion, the upper portion defining a cutting slot between a preserved side and a sacrificial side, the head configured to hold a nerve in tension while circumferentially constraining a portion of the nerve on the preserved side. The nerve cutter may also comprise a body connected to the upper portion and the lower portion of the head, the body forming a hinge to facilitate holding the nerve in tension by the upper portion and the lower portion.

The nerve cutter may also comprise any of the foregoing in any combination:
a blade capable of fitting into the cutting slot.
the blade comprises a scalpel blade.
the sacrificial side comprises a rough finish.
the sacrificial side comprises a surface having an anti-slip surface material.
a plurality of guides configured to measure a plurality of sized nerves.
first and second guides, wherein the first guide is configured to measure a first size of a nerve and the second guide is configured to measure a second size of a nerve, wherein the first size is greater than the second size.
the hinge is monolithically formed with the body.

A nerve cutter configured to hold a nerve in tension while circumferentially constraining a side of the nerve being retained in a manner that prevents flattening or crushing may comprise a head configured to separate a preserved side of the nerve from a portion of the nerve being cut, where the head comprises a cutting slot configured to accommodate a scalpel blade, where the cutting slot permits the scalpel blade to remain approximately square.

The nerve cutter may also comprise any of the foregoing in any combination:
a side of the head that interfaces with the preserved side of the nerve is configured to prevent and/or inhibit the nerve from moving radially to its axis while being cut.
the side comprises a rough finish.
the side comprises a surface having an anti-slip surface material.
a plurality of guides configured to measure a plurality of sized nerves.

A nerve cutter may comprise a head having an upper portion and a lower portion that meet at a back stop, the upper portion defining a cutting slot between a preserved side and a sacrificial side, the head configured to hold a nerve in tension while circumferentially constraining a portion of the nerve on the preserved side and the back stop configured to laterally constrain the nerve. The nerve cutter may also comprise a body connected to the upper portion and the lower portion of the head, the body forming a hinge to facilitate holding the nerve in tension by the upper portion and the lower portion.

The nerve cutter may also comprise any of the foregoing in any combination:
the cutting slot is sized to operatively accept a blade of between 0.15 mm to 0.75 mm.
the upper portion and lower portion of the head join at a hinge.
the hinge is monolithically formed with the upper portion and lower portion of the head.
the upper portion comprises an upper nerve interface.
the lower portion comprises a lower nerve interface.
a first guide of a size configured to engage a nerve of a first size.
a second guide of a size configured to engage a nerve of a second size, wherein the first size is larger than the second size.

These and other features and advantages of the present disclosure are set forth in the following specification, drawings and claims.

### BRIEF DESCRIPTION

Operation of the disclosure may be better understood by reference to the following detailed description taken in connection with the following illustrations, wherein:

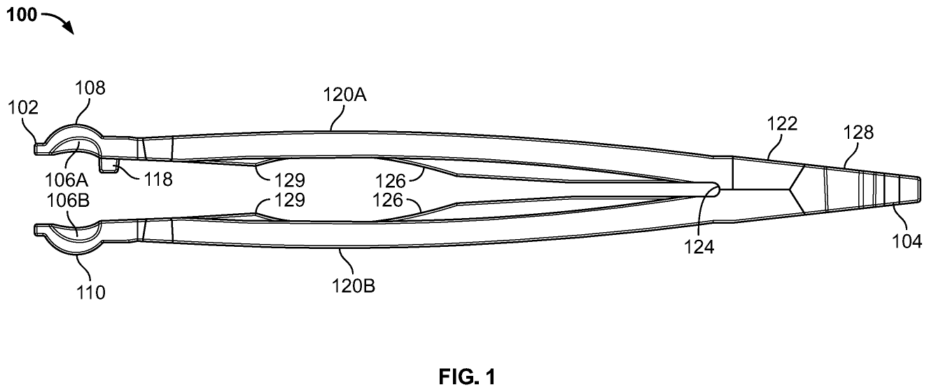

FIG. 1 is a side view of a circumferential nerve cutter, in accordance with the teachings of this disclosure.

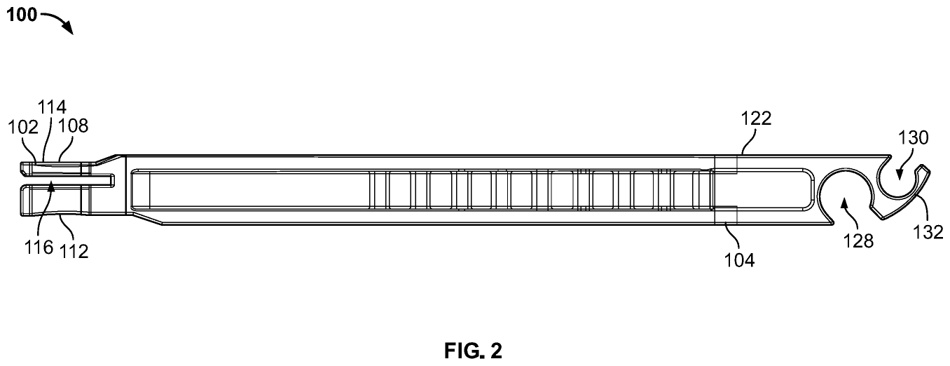

FIG. 2 is a top view of the circumferential nerve cutter, in accordance with the teachings of this disclosure.

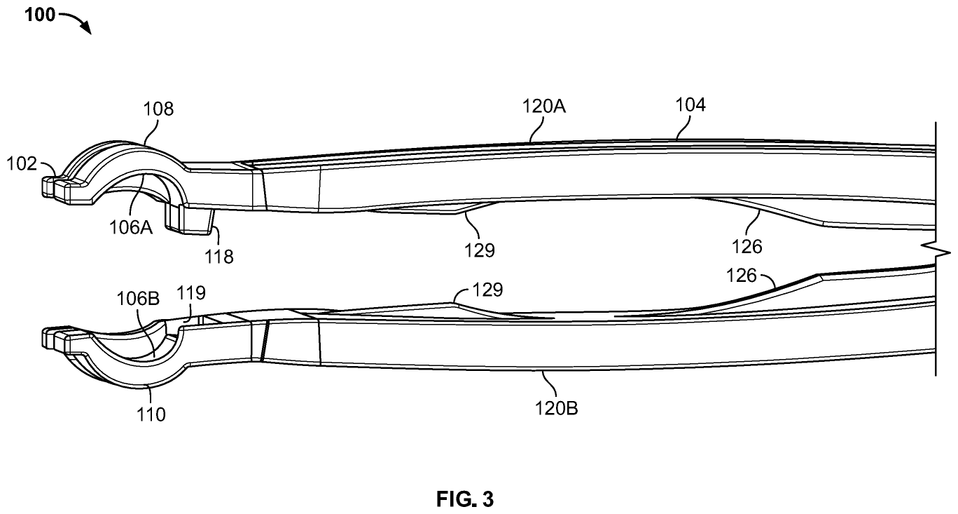

FIG. 3 is a perspective view of the circumferential nerve cutter, in accordance with the teachings of this disclosure.

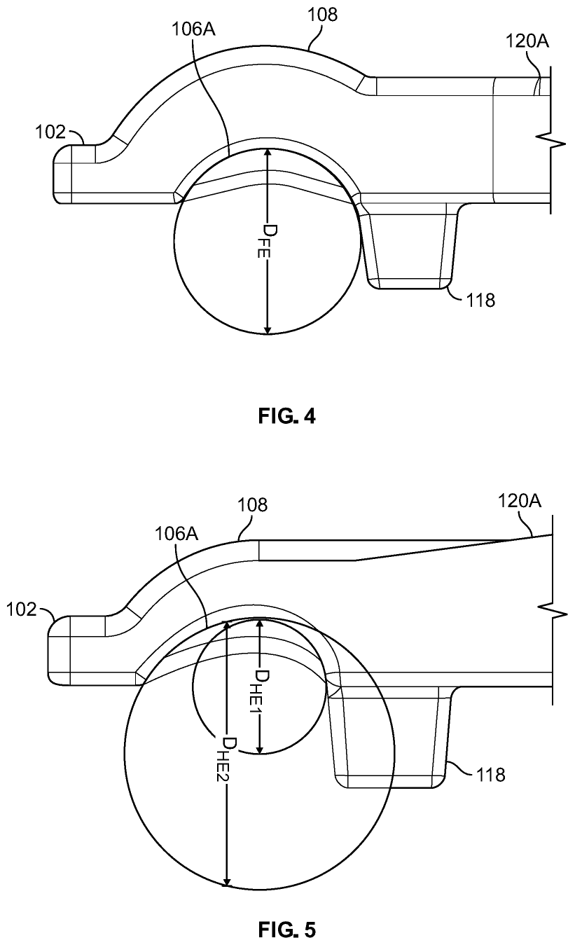

FIG. 4 is a side view of an example head of the circumferential nerve cutter, in accordance with the teachings of this disclosure.

FIG. 5 is a side view of another example head of the circumferential nerve cutter, in accordance with the teachings of this disclosure.

### DESCRIPTION

Reference will now be made in detail to exemplary embodiments of the present disclosure, examples of which are illustrated in the accompanying drawings. It is to be understood that other embodiments may be utilized, and structural and functional changes may be made without departing from the respective scope of the disclosure set forth herein. Moreover, features of the various embodiments may be combined or altered without departing from the scope of the present disclosure. As such, the following description is presented by way of illustration only and should not limit in any way the various alternatives and modifications that may be made to the illustrated embodiments and still be within the spirit and scope of the invention.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

It is noted that the various embodiments described herein may include other components and/or functionality. It is further noted that while various embodiments refer to a nerve cutter, various other systems may be utilized in view of embodiments described herein. For example, embodiments may be utilized in a variety of surgical procedures. As such, embodiments may refer to a particular surgical procedure for purposes of explanation. It is noted that aspects of embodiments, however, may be utilized for various other procedures.

This disclosure generally relates to systems and methods that may improve nerve dissection or cutting such that the applicable nerve may have performed a coaptation. Upon such the coaptation nerve may have applied thereto regeneration or neuroregeneration via electrical stimulation to increase the speed or amount of nerve growth. The terms “nerve” or “nerve tissue” generally refer to any portion of a nerve including, but not limited to, axons, axon terminals, somas, dendrites, or the like, unless context suggest otherwise. Moreover, aspects disclosed herein may be applicable to nerve tissue throughout a body, whether peripheral nervous tissue or otherwise. Further, while embodiments may reference a surgeon performing a particular action(s), it is noted that other users, automated machines, or the like may perform such actions.

It is noted that described systems and methods may be utilized in combination with various systems and methods for safeguarding against nerve injury during surgical procedures, such as coaptation of the nerve. The systems and methods are particularly well suited for being utilized in conjunction with a device that may be utilized by a surgeon in identification of nerves and muscles in order to assure nerve integrity during such medical procedures as well as during a nerve regeneration process such as that disclosed in U.S. patent Ser. No. 16/623,538, which is incorporated herein by reference.

For example, a surgeon may utilize the present nerve cutter to perform a dissection of the applicable nerve, perform a coaptation of the nerve and use a handheld stimulation device to generate a stimulation signal at sufficiently high levels for the purposes of locating, stimulating, and evaluating nerve integrity in numerous medical procedures, including, but not limited to, evaluating nerve integrity to determine if a further repair may be needed, identifying specific nerve branches or fascicles for repair, and evaluating nerve function following a repair prior to completing a surgical procedure. In addition or in the alternative, before, after or during a procedure, a surgeon may place an electrode or lead on or near the nerve to be stimulated such as that one that was just cut using the nerve cutter of the present disclosure and/or proximal to the site of injury or repair. The electrode may be percutaneous or non-percutaneous (e.g., surface electrode). In an aspect, a percutaneous lead may be taped or otherwise held in place on a patient's skin. This may allow for easy removal after the stimulation such as that applied for nerve regeneration.

FIGS. 1-3 illustrate a circumferential nerve cutter 100 configured to hold a nerve in tension while circumferentially constraining the preserved side of the nerve to reduce the likelihood of crushing the nerve or making uneven cuts in the nerve. In the illustrated example, the circumferential nerve cutter 100 includes a head 102 and a body 104. The head 102 defines an upper nerve interface 106A and a lower nerve interface 106B (collectively “nerve interface 106”). As best seen in FIG. 1, the head 102 comprises an upper member 108 and a lower member 110.

As best seen in FIG. 2, the circumferential nerve cutter 100 has a preserved side 112 (e.g., the side that grips the side of the nerve to remain) and a sacrificial side 114 (e.g., the side that grips the side of the nerve to be removed). The head 102 defines a cutting slot 116 between the preserved side 112 and the sacrificial side 114. As illustrated in FIGS. 1 and 3, the upper member 108 of the head 102 includes a backstop 118 to laterally constrain the nerve to be cut. As shown in FIG. 3, the lower member 110 of the head 102 includes a well 119 that corresponds with the backstop 118, which allows each portion of the head 102 on the upper arm 120A and lower arm 120B to sit flush with one another.

The head 102 separates the preserved side and the sacrificial side of the nerve being cut. The cutting slot 116 separates, at least partially, the preserved side 112 and the sacrificial side 114 of the head 102. The width of the cutting slot 116 is configured to, for example, accommodate a surgical cutting blade of any configuration. In some examples, the width of the cutting slot 116 may be configured for a specific size of blade. For example, the width of the cutting slot 116 may accommodate a 0.15 millimeter (mm) blade. In some examples, the width of the cutting slot 116 may be configured for the widest applicable blade (e.g., to accommodate up to a 0.75 mm for a scalpel blade, etc.) The shape of the cutting slot 116 is configured to ensure or promote the blade remaining approximately square (i.e., within 15 degrees of square) preventing obliquity of a resultant cut.

The sacrificial side 114 of the head 102 may be configured to grip the sacrificial end of the nerve being cut with additional material creating an aperture smaller than the preserved side 112 of the head 102. This allows tension along the length of the nerve, lateral constraint (e.g., via the backstop 118), and circumferential constraint (e.g., to prevent flattening, etc.) to be applied during the cutting process. This allows for a more precise and straighter cut without crushing the intended preserved side of the nerve. Further, it allows for a more repeatable cut that is consistent between each of the cuts performed. The sacrificial side 114 of the head 102 may grip the nerve through compression by including a surface of an anti-slip, high coefficient of friction material or surface or any kind of rough finish to assist in holding the nerve to prevent it from moving during a dissection when utilizing the nerve cutter 100. In some embodiments, the sacrificial side 114 of the head 102 may comprise a texturing of the nerve interface 106, spikes along the nerve interface 106, knurling of the nerve interface 106, and/or by incorporation of any kind of surface finish along the nerve interface 106. The sacrificial side aperture may also be configured to be identical in opening to the preserved side 112 aperture. The circumferential constraint on the preserved side 112 of the head 102 may be configured to allow the nerve to be constrained from flattening, reshaping, or being crushed while being cut, but not to prevent the nerve from sliding axially out of the circumferential nerve cutter 100.

The nerve interface 106 of the preserved side 112 is configured to prevent or inhibit the nerve from moving radially to its axis while being cut. The nerve interface 106 and the backstop 118 facilitate the circumferential nerve cutter 100 being used across a range of nerve diameters (e.g. from 1-3 mm, from 3-5 mm or from 5-7 mm, etc.) without the nerve sliding toward the body 104. In the illustrated example of FIG. 4, the nerve interface 106 and the backstop 118 is a defined diameter (D_{FE}) that corresponds with the diameter for the largest size nerve the circumferential nerve cutter 100 is configured to accommodate. In the illustrated example of FIG. 5, a portion of the nerve interface 106 and the backstop 118 define a small diameter (D_{HE1}) that is the diameter of the smallest nerve to be accommodated and a large diameter (D_{HE2}) that is the diameter of the largest nerve to be accommodated. The present teachings are not limited to just those dimensions disclosed herein, but any dimension may be accommodating through the teachings disclosed herein.

The body 104 may include an upper arm 120A and a lower arm 120B (collectively referred to as “arms” 120) connected by an end portion 122 at a hinge 124 by any appropriate manner, such as being monolithically formed, fixed together through welding, fastening, adhesives or the like. The upper arm 120A is connected to the upper member 108 of the head 102 such as being monolithically formed therewith or fixed together through welding, fastening, adhesives or the like. The lower arm 120A is connected to the lower member 110 of the head such as being monolithically formed therewith or fixed together through welding, fastening, adhesives or the like. The arms 120 may be integrally formed with their corresponding members 108 and 110, as well as the end portion 122. For example, the circumferential nerve cutter 100 may be molded or otherwise manufactured from one piece, i.e., it may be monolithically formed.

Alternatively, the arms 120 may be fixed together with their corresponding members 108 and 110 such as through welding, fastening, adhesives or the like. The material of the circumferential nerve cutter 100 may be flexible enough and the hinge 124 may be configured to facilitate bending of the arms 120 relative each other. The arms 120 may each include stops 126 that limit the movement of the arms 120 relative each other. The arms 120 may also include ribs 129 that may also limit movement of the arms 120 relative each other. The stops 126 and ribs 129 are configured to facilitate bending of the arms 120 such that the nerve interface 106 grips the nerve to provide lateral and circumferential constraint to the nerve. Further, the stops 126 and ribs 129 may be configured to provide support or stiffening to the body 104—the stops 126 and ribs 129 may be stiffeners. In some examples, an outer surface of the arms 120 may be textured to facilitate gripping the circumferential nerve cutter 100 by the user thereof.

As best seen in FIG. 2, the body 104 defines guides 128 and 130. In the illustrated example, the guides 128 and 130 are substantially perpendicular (i.e., within two five degrees of perpendicular or in some embodiment 15 degrees of perpendicular) to the nerve interface 106. The guide cutouts, 128 and 130, are configured to facilitate selection of the appropriate cutter from a range of size offerings by defining the smallest and largest diameter that the nerve cutter 100 can operatively grasp to cut. The portion of the nerve cutter 100 containing the guide cutouts 128 and 130 may be configured to minimize the thickness of the end portion 122 and placed in a location to enable the sizing operation while requiring minimal lifting of the nerve within the surgical wound. The guide cutout 130 that defines the smallest size is at the end of the body 104. The guide cutout 128 that defines the largest size is located inside proximate the smaller guide cutout 130, stemming off a curved edge 132, which allows the user to lift the nerve minimally if the nerve is in continuity. The guide cutouts 128 and 130 have rounded edges to facilitate a hooking under motion to size the nerve.

The circumferential nerve cutter 100 may be formed of any applicable material. In one exemplary embodiment, the circumferential nerve cutter 100 may be formed from plastic or rubber of any configuration. The circumferential nerve cutter 100 may also be formed from a metal, such as aluminum. Further, the circumferential nerve cutter 100 may be disposable such that it is packaged in a sterilized container such that the circumferential nerve cutter 100 may be removed from its package and be ready for use in surgery or the applicable procedure. The circumferential nerve cutter 100 may be formed of a material that is capable of being sterilized so that the circumferential nerve cutter 100 can be sterilized, such as being put in an autoclave, after each use. Further still, the circumferential nerve cutter 100 being monolithically formed may make it to be capable of being sterilized after each use, i.e., there are no attachment points by which matter can be trapped.

Although the embodiments of this disclosure have been illustrated in the accompanying drawings and described in the foregoing detailed description, it is to be understood that the present disclosure is not to be limited to just the described embodiments, but that the embodiments described herein are capable of numerous rearrangements, modifications and substitutions without departing from the scope of the claims hereafter. It is, of course, not possible to describe every conceivable combination of components or methodologies for purposes of describing the present specification, but one of ordinary skill in the art may recognize that many further combinations and permutations of the present specification are possible. Each of the components described above may be combined or added together in any permutation to define an introducing device and/or introducing system. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim. The claims as follows are intended to include all modifications and alterations insofar as they come within the scope of the claims or the equivalent thereof.

1. A nerve cutter comprising:
a head having an upper portion and a separate lower portion, the upper portion defining a cutting slot between a preserved side and a sacrificial side, the head configured to hold a nerve in tension while circumferentially constraining a portion of the nerve on the preserved side; and
a body connected to the upper portion and the lower portion of the head, the body forming a hinge to facilitate holding the nerve in tension by the upper portion and the lower portion.

2. The nerve cutter of claim 1 further comprising a blade capable of fitting into the cutting slot.

3. The nerve cutter of claim 2, wherein the blade comprises a scalpel blade.

4. The nerve cutter of claim 1, wherein the sacrificial side comprises a rough finish.

5. The nerve cutter of claim 1, wherein the sacrificial side comprises a surface having an anti-slip surface material.

6. The nerve cutter of claim 1 further comprising a plurality of guides configured to measure a plurality of sized nerves.

7. The nerve cutter of claim 1 further comprising first and second guides, wherein the first guide is configured to measure a first size of a nerve and the second guide is configured to measure a second size of a nerve, wherein the first size is greater than the second size.

8. The nerve cutter of claim 1, wherein the hinge is monolithically formed with the body.

9. A nerve cutter configured to hold a nerve in tension while circumferentially constraining a side of the nerve being retained in a manner that prevents flattening or crushing, the nerve cutter comprising:
a head configured to separate a preserved side of the nerve from a portion of the nerve being cut, wherein the head comprises a cutting slot configured to accommodate a scalpel blade, wherein the cutting slot permits the scalpel blade to remain approximately square.

10. The nerve cutter of claim 9, wherein a side of the head that interfaces with the preserved side of the nerve is configured to prevent and/or inhibit the nerve from moving radially to its axis while being cut.

11. The nerve cutter of claim 10, wherein the side comprises a rough finish.

12. The nerve cutter of claim 1, wherein the side comprises a surface having an anti-slip surface material.

13. The nerve cutter of claim 9 further comprising a plurality of guides configured to measure a plurality of sized nerves.

14. A nerve cutter comprising:
a head having an upper portion and a lower portion that meet at a back stop, the upper portion defining a cutting slot between a preserved side and a sacrificial side, the head configured to hold a nerve in tension while circumferentially constraining a portion of the nerve on the preserved side and the back stop configured to laterally constrain the nerve; and
a body connected to the upper portion and the lower portion of the head, the body forming a hinge to facilitate holding the nerve in tension by the upper portion and the lower portion.

15. The nerve cutter of claim 14, wherein the cutting slot is sized to operatively accept a blade of between 0.15 mm to 0.75 mm.

16. The nerve cutter of claim 14, wherein the upper portion and lower portion of the head join at a hinge.

17. The nerve cutter of claim 16, wherein the hinge is monolithically formed with the upper portion and lower portion of the head.

18. The nerve cutter of claim 14, wherein the upper portion comprises an upper nerve interface.

19. The nerve cutter of claim 18, wherein the lower portion comprises a lower nerve interface.

20. The nerve cutter of claim 14 further comprising a first guide of a size configured to engage a nerve of a first size.

21. The nerve cutter of claim 20 further comprising a second guide of a size configured to engage a nerve of a second size, wherein the first size is larger than the second size.
